(19) United States
(12) Patent Application Publication
(10) Pub. No.: US 2013/0289522 A1
(43) Pub. Date: Oct. 31, 2013

(54) Methods and Systems for Closed Loop Neurotrophic Delivery Microsystems

(71) Applicant: The Royal Institution for the Advancement of Learning / McGill University, Montreal (CA)

(72) Inventors: Wissam Sam Musallam, Montreal (CA); Mohammad Pousinchi, Montreal (CA)

(21) Appl. No.: 13/869,160

(22) Filed: Apr. 24, 2013

Related U.S. Application Data
(60) Provisional application No. 61/637,320, filed on Apr. 24, 2012.

(51) Int. Cl. A61M 5/172

(52) U.S. Cl. CPC A61M 5/1723
USPC 604/503

(57) ABSTRACT

Brain Machine Interfaces (BMIs) promise to improve the lives of many patients by providing a direct communication pathway between the brain and one or more external devices. As the brain is an electrochemical system additional signals may improve BMI performance beyond direct electrical signals. Further many psychiatric and neurological disorders such as Parkinson's disease, depression, dystonia, or obsessive compulsive disorder are related to neurotransmitter deficiencies or imbalances. Accordingly detection of neurotransmitter chemicals and/or management of these chemicals may enhance BMIs. Embodiments of the invention provide for implantable CMOS based target derived neurotrophic factor delivery microsystems and neurochemical sensors allowing neurotransmitter deficiencies or imbalances to be detected, monitored, and corrected. Such implantable CMOS solutions provide for high volume, low cost manufacturing as well integration options in arrayed formats as well as integration with other CMOS electronic circuits.

### CROSS-REFERENCE TO RELATED APPLICATIONS

This patent application claims the benefit of U.S. Provisional Patent Application U.S. 61/637,320 filed Apr. 24, 2012 entitled “Methods and Systems for Closed Loop Neurotrophic Delivery Microsystems”, the entire contents of which are included by reference.

### FIELD OF THE INVENTION

The present invention relates to CMOS implantable electronics and more specifically to neurochemical sensors and neurotrophic factor delivery microsystem.

### BACKGROUND OF THE INVENTION

Brain Machine Interfaces (BMIs) promise to improve the lives of many patients by providing a direct communication pathway between the brain and one or more external devices. Action Potential and Local Field Potential electrophysiological signals have been shown to contain viable information for controlling prosthetic devices, see for example Olanow et al “Continuous dopamine-receptor treatment of Parkinson's disease: scientific rationale and clinical implications” (The Lancet Neurology, Vol. 5(8), pp 677-687); Rascol et al “A five-year study of the incidence of dyskinesia in patients with early Parkinson's disease who were treated with ropinirole or levodopa” (New England J. of Medicine, Vol. 342(20), pp 1484-1491); Buck et al “L-DOPA-induced dyskinesia in Parkinson's disease: a drug discovery perspective” (Drug Discovery Today); Gross “Deep brain stimulation in the treatment of neurological and psychiatric disease.” (Expert Rev. Neurotherapeutics, Vol. 4(3), pp 465-478) and Derost et al “Is DBS-STN appropriate to treat severe Parkinson disease in an elderly population?” (Neurology, Vol. 68(17), 1345). However, the brain is an electrochemical system and contains additional signals that may improve BMI performance. Action potentials are initiated by the release of neurotransmitters from presynaptic neurons. Many psychiatric and neurological disorders such as Parkinson's disease, depression, dystonia, or obsessive compulsive disorder are related to neurotransmitter deficiencies or imbalances, see for example Santens et al. “Lateralized effects of subthalamic nucleus stimulation on different aspects of speech in Parkinson's disease” (Brain and Language, Vol. 87(2), pp 253-258); Benarroch “Subthalamic nucleus and its connections” (Neurology, Vol. 70(21)); and Barker “Parkinson's disease and growth factors-are they the answer?” (Parkinsonism & Related Disorders, Vol. 15, S181-S184). Detection of these chemicals may therefore carry additional information that can be used to enhance BMI performance.

Considering Parkinson's disease (PD) this is the second most widespread neurodegenerative disorder after Alzheimer's disease. In 2005 between 4.1 and 4.6 million individuals were diagnosed with PD and based on scientific predictions this number will increase to 8.7 to 9.3 million by 2030. PD is caused by the depletion of dopamine in the striatum due to death of dopaminergic neurons in the substantia nigra. At present the main treatment for PD is pharmacological dopamine replacement within the nigra-stratum region. This replacement can occur by administration of the L-dopa (L-3,4-dihydroxyphenylalanine) which is a dopamine precursor and the most widely used medicine for the treatment of PD. Although this method improves the patient's condition remarkably it does not lead to restoration of damaged dopaminergic neurons or protection of those remaining.

Additionally, after a few years of L-dopa therapy, the majority of patients experience serious side effects such as the “on-off” effect wherein patients can move during “on” period and they are completely immobile during the “off” period. Moreover, a subset of patients suffer from L-dopa induced dyskinesias during “on” periods. An alternative therapy which has emerged as a breakthrough in PD treatment is Deep Brain Stimulation (DBS) wherein in this therapeutic method an implanted electrode continuously delivers 3-5 Volt pulses approximately 0.1 ms wide at 100 Hz to the sub-thalamic nucleus. Stimulation of the sub-thalamic nucleus has been proven to be highly effective at reducing various PD symptoms, see Derost et al “Is DBS-STN appropriate to treat severe Parkinson disease in an elderly population?” (Neurology, Vol. 68(17), pp 134-5). However, DBS can lead to speech impairment, cognitive maladjustment, psychological dysfunction, and other co-morbid conditions. Additionally current leakage into adjacent nuclei can also lead to uncomfortable sensations for the patient. Whilst these side effects may be ameliorated by reducing the stimulation amplitude this comes at the cost of reduction in DBS efficacy.

Regardless of these side effects, pharmacological treatment and DBS remain the two major therapeutic methods for Parkinson's disease. Over the past 30 years several interesting approaches for PD treatment have been emerged where the main goal of these methods is to restore or replace the damaged dopaminergic neurons and provide neuroprotection for remaining ones. These major restorative therapies include cell transplantation, dopaminergic neuron derivation from embryonic stem cells, neurogenesis and the direct delivery of nerve growth factor to the brain. Each treatment has its own advantages and disadvantages. For instance, in embryonic cell transplantation, the shortage of donor tissue is the most important limiting factor and less than 20% of these cells survive transplantation. Whilst all these techniques are invasive in approach nerve growth factor offers advantages in that it may be employed pre-emptively (for protection and/or early treatment) and does not require consideration of how to address the human body's immune response to the introduction of foreign tissue or materials.

The protection and regeneration of dopaminergic neurons in Parkinson's disease requires that glial cell line-derived neurotrophic factor (GDNF) be directly delivered into the striatum, see for example Jollivet et al “Striatal implantation of GDNF releasing biodegradable microspheres promotes recovery of motor function in a partial model of Parkinson's disease” (Biomaterials, Vol. 25(5), pp 933-942); Aoi et al “Single or continuous injection of glial cell line-derived neurotrophic factor in the striatum induces recovery of the nigrostriatal dopaminergic system” (Neurological Research, Vol. 22(8), pp 832), Popovic et al “Therapeutic potential of controlled drug delivery systems in neurodegenerative diseases” (Int. J. Pharmaceutics, Vol. 314(2), pp 120-126); Bilang-Bleuel et al “Intrastriatal injection of an adenoviral vector expressing glial-cell-line-derived neurotrophic factor prevents dopaminergic neuron degeneration and behavioral impairment in a rat model of Parkinson disease” Proc. Nat. Ass. Sci. USA, Vol. 94(16), pp 8818); Park et al “Protection of nigral neurons by GDNF-engineered marrow cell transplantation” (Neuroscience Res., Vol. 40(4), pp 315-323); and Kishima et al “Encapsulated GDNF-producing C2C12 cells for Parkinson's disease: a pre-clinical study in chronic MPTP-treated baboons” (Neurobiology of Disease, Vol. 16(2), pp 428-439). It has been shown by several clinical trials and preclinical studies that GDNF's neuroprotective and regeneration effects for dopaminergic neurons exceed other neurotrophic factors, see for example Alexi et al “Neuroprotective strategies for basal ganglia degeneration: Parkinson's and Huntington's Diseases.” (Progress in Neurobiology, Vol. 60(5), pp 409-470) and Gash et al in “Neuroprotective and neurorestorative properties of GDNF” (Annals of Neurology, Vol. 44(3 Suppl 1), S121). There are several intracranial GDNF administration strategies available and some important achievements obtained by enforcing these methods in open-label clinical trials, see Gill et al. “Direct brain infusion of glial cell line-derived neurotrophic factor in Parkinson disease” (Nature Medicine, Vol. 9(5), pp 589-595) and Slevin et al “Improvement of bilateral motor functions in patients with Parkinson disease through the unilateral intraputaminal infusion of glial cell line-derived neurotrophic factor” (J. Neurosurgery, Vol. 102(2), pp 216-222).

However, these administration strategies face a number of limitations including for example a lack of control over infusion rate, see Gill, and GDNF dosage, see Saltzman et al. “Intracranial delivery of recombinant nerve growth factor: release kinetics and protein distribution for three delivery systems” (Pharm. Res., Vol. 16(2), pp 232-240) and Jollivet et al. “Striatal implantation of GDNF releasing biodegradable microspheres promotes recovery of motor function in a partial model of Parkinson's disease” (Biomaterials, Vol. 25(5), pp 933-942), strong immune system response, see Choi-Lundberg et al “Dopaminergic neurons protected from degeneration by GDNF gene therapy” (Science, Vol. 275(5301), 838) and Choi-Lundberg et al. “Behavioral and Cellular Protection of Rat Dopaminergic Neurons by an Adenoviral Vector Encoding Glial Cell Line-Derived Neurotrophic Factor* 1” (Exp. Neurology, Vol. 154(2), pp 261-275) in addition to accidental insertional mutagenesis in gene therapy, see for example Hacein-Bey-Abina et al. “LMO2-associated clonal T cell proliferation in two patients after gene therapy for SCID-X1” (Science, Vol. 302(5644), 415) and Li et al. “Murine leukemia induced by retroviral gene marking” (Science, Vol. 296(5567), 497).

In order to mitigate some of these limitations the inventors have addressed the fact that current GDNF administration strategies are based on open-loop systems. In order to control the infusion rate and GDNF dosage, having a negative feedback closed loop system such as described in respect of FIG. 1 corrects for this. Accordingly, the delivery microsystem obtains information from the environment (substantia nigra) and based on the collected data the delivery microsystem can not only control the infusion rate and but determined what GDNF dosage is required. Accordingly, sensor electrodes 120 and optical sensors 130 provide measurements of predetermined chemicals resulting from neurochemical processes within the brain 110. The outputs of these sensors are coupled to sensing circuit 140 which provides amplification and integration as well as other signal processing functions as required. The output from sensing circuit 140 is coupled to decision making circuit 150 which is interfaced to microfluidic pump and neurotrophic factor delivery system 160 which under control signals provided from the decision making circuit 150 provides controlled dosage of drug(s), such as GDNF for example.

Accordingly, it would be beneficial to provide an implantable CMOS based target derived neurotrophic factor delivery microsystem (NEUFADEMS) 200 such as depicted in respect of FIG. 2 wherein a silicon micromachined structure 210 which comprises on a first side a sensor 220 which is coupled to CMOS electronics 240 via electrical interconnect 230. On the other side of silicon micromachined structure 210 a microfluidic drug reservoir 260 is connected to dispensing locations 280 via microfluidic channel 270. Such a NEUFADEMS 200 according to embodiments of the invention may maintain therapeutic levels of dopamine concentrations in the brain in order to protect healthy neurons and restore damaged ones. Such an implantable intelligent microsystem senses the depletion of dopamine in nigrostraital pathway(s) using a novel sensor and sensing CMOS circuit which is able to sense micro-molar concentration of dopamine. Then, by means of a negative feedback loop the NEUFADEMS may control the flow of GDNF within micro-fluidic channels such that microelectromechanical (MEMS) pumps which are connected to the microfluidic channels on the probe may inject micro-molar concentrations of neurotrophic factor into the brain.

It would be beneficial therefore for such a NEUFADEMS to exploit CMOS electronics for low power consumption, integration with the micro-fluidic delivery system, and MEMS integration within a common silicon substrate. According to a first embodiment of the invention the inventors provide a sensing, control and decision making circuit for such a NEUFADEMS. It consists of a Current Conveyer, a low noise low power amplifier, an integrator and a comparator with offset cancelation and is compatible with standard silicon CMOS processing. Implemented in 0.18 μm CMOS an embodiment of the invention yields a circuit consuming only 921 nW whilst maintaining a bandwidth of 2.75 kHz.

In order to detect and measure the very low signals from neurotransmitters, a highly sensitive device such as potentiostat is needed. Potentiostats generate an electrochemical current that is proportional to the chemical concentration around the electrodes as shown in FIG. 3. However, prior art potentiostats are typically not suitable for in vivo neurotransmitter recording applications as they are typically laboratory instruments with poor sensitivity as generally designed for large chemical concentration measurements resulting in currents of microamps to milliamps. Additionally as laboratory instruments they are generally large, heavy and very expensive.

Accordingly it would be beneficial for a neurochemical sensor to not only minimize power consumption and the microsystem's noise but also provide a low cost solution unlike potentiostats. The inventors have established an implantable low power low noise CMOS neurochemical sensor which is able to sense micro-molar concentration of different neurotransmitters such as dopamine and serotonin. The sensing component of the device consists of a reference, counter and working electrode connected to low noise low power integrator amplifier and a current mode 10-bit first order sigma delta Analog to Digital Converter (ADC). It converts the measured red-ox current (picoscale to microscale) to digital codes for further processing. A neurochemical sensor according to an embodiment of the invention consumes 120.85 μW and provides low input referred noise (transistor noise).

Accordingly, embodiments of the invention provide for implantable CMOS based target derived NEUFADEMS and implantable CMOS neurochemical sensors allowing neurotransmitter deficiencies or imbalances to be detected, monitored, and corrected. Such implantable CMOS solutions provide for high volume, low cost manufacturing as well integration options in arrayed formats as well as integration with other CMOS electronic circuits including for example microprocessors, microcontrollers, static random access memory, other digital logic circuits, analog circuits, and mixed digital/analog circuits. Beneficially such low cost high performance CMOS circuit solutions may be employed in the management of many psychiatric and neurological disorders including, but not limited to, Parkinson's disease, depression, dystonia, and obsessive compulsive disorder.

Other aspects and features of the present invention will become apparent to those ordinarily skilled in the art upon review of the following description of specific embodiments of the invention in conjunction with the accompanying figures.

### SUMMARY OF THE INVENTION

It is an object of the present invention to mitigate disadvantages in the prior art relating to CMOS implantable electronics and more specifically to neurochemical sensors and NEUFADEMS.

In accordance with an embodiment of the invention there is provided a method comprising
determining a concentration of a neurotransmitter in-situ using an electrochemical sensor integrated into a probe;
coupling the output of the electrochemical sensor to a CMOS processing circuit integrated with the probe, the CMOS processing circuit providing an output in determination of at least the output of the electrochemical sensor and a reference;
coupling the output of the CMOS processing circuit to a microfluidic delivery system integrated within the probe, the microfluidic delivery system providing localized delivery of a predetermined drug in dependence upon the output of the CMOS processing circuit.

In accordance with an embodiment of the invention there is provided a method comprising maintaining a neurotransmitter above a predetermined concentration with a predetermined region of a brain using a closed-loop neurotrophic factor delivery and control system.

In accordance with an embodiment of the invention there is provided a device comprising
an electrochemical sensor for determining a concentration of a neurotransmitter;
a CMOS processing circuit electrically coupled to the electrochemical sensor providing an output in determination of at least the output of the electrochemical sensor;
a microfluidic delivery system coupled to the CMOS processing circuit for providing localized delivery of a predetermined drug in dependence upon the output of the CMOS processing circuit.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 depicts a system level block diagram of an Implantable Intelligent CMOS Neurotrophic factor Delivery Microsystem according to an embodiment of the invention;

FIG. 2 depicts a 3D view of an Implantable Intelligent CMOS Based Neurotrophic factor Delivery Microsystem according to an embodiment of the invention;

FIG. 3 depicts a schematic of the electro analysis setup according to an embodiment of the invention;

FIG. 4 depicts a circuit schematic of a Sensing and Control Circuit for an Implantable Intelligent CMOS Based Neurotrophic factor Delivery Microsystem according to an embodiment of the invention;

FIG. 5 depicts a Wide Swing Folded Cascade Circuit for an Implantable Intelligent CMOS Based Neurotrophic factor Delivery Microsystem according to an embodiment of the invention;

FIG. 6 depicts a Latched Comparator with Offset Cancelation Circuit for an Implantable Intelligent CMOS Based Neurotrophic factor Delivery Microsystem according to an embodiment of the invention;

FIG. 7 depicts a Latched Comparator with Offset Cancelation Circuit for an Implantable Intelligent CMOS Based Neurotrophic factor Delivery Microsystem according to an embodiment of the invention;

FIG. 8 depicts Op-Amp AC analysis results for an Op-Amp forming part of a current conveyor for an Implantable Intelligent CMOS Based Neurotrophic factor Delivery Microsystem according to an embodiment of the invention;

FIG. 9 depicts Microsystem Transient Analysis results for an Implantable Intelligent CMOS Based Neurotrophic factor Delivery Microsystem according to an embodiment of the invention;

FIG. 10 depicts experimental results for an Implantable Intelligent CMOS Based Neurotrophic factor Delivery Microsystem according to an embodiment of the invention;

FIG. 11 depicts a System-Level Chip schematic of an Implantable CMOS Neurochemical Sensor according to an embodiment of the invention;

FIG. 12 depicts a 1st Order Sigma Delta ADC system level schematic for use within an Implantable CMOS Neurochemical Sensor according to an embodiment of the invention;

FIG. 13 depicts a 1st Order Sigma Delta ADC circuit schematic for use within an Implantable CMOS Neurochemical Sensor according to an embodiment of the invention;

FIG. 14 depicts a Front End for a microsystem forming part of an Implantable CMOS Neurochemical Sensor according to an embodiment of the invention;

FIG. 15 depicts a PSD Plot for 10-bit First order Sigma Delta ADC forming part of an Implantable CMOS Neurochemical Sensor according to an embodiment of the invention;

FIG. 16 depicts the Static Red-Ox Current in Response to Addition of 5 μM Dopamine for an Implantable CMOS Neurochemical Sensor according to an embodiment of the invention;

FIG. 17 depicts the Current Transfer Characteristics of an Implantable CMOS Neurochemical Sensor according to an embodiment of the invention;

FIG. 18 depicts an exemplary manufacturing process according to an embodiment of the invention;

FIG. 19A through 19I depict an exemplary probe configuration comprising a neurotrophic factor delivery microsystem according to an embodiment of the invention in conjunction with an optoelectronic sensor and electronic stimulation and neurochemical measurement circuits;

FIG. 20 depicts an exemplary probe configuration comprising a neurotrophic factor delivery microsystem according to an embodiment of the invention in conjunction with an optoelectronic sensor and electronic stimulation and neurochemical measurement circuits; and

FIG. 21 depicts an exemplary probe configuration comprising a neurotrophic factor delivery microsystem according to an embodiment of the invention in conjunction with an optoelectronic sensor and electronic stimulation and neurochemical measurement circuits.

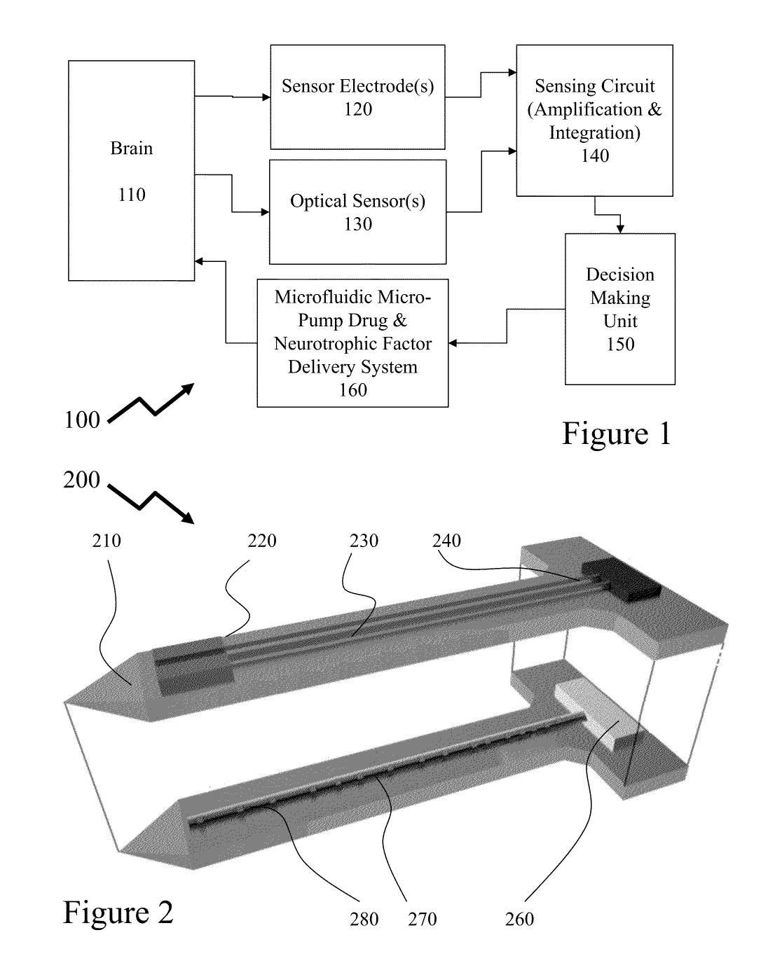

### DETAILED DESCRIPTION

The present invention is directed to CMOS implantable electronics and more specifically to neurochemical sensors and neurotrophic factor delivery microsystems.

The ensuing description provides exemplary embodiment(s) only, and is not intended to limit the scope, applicability or configuration of the disclosure. Rather, the ensuing description of the exemplary embodiment(s) will provide those skilled in the art with an enabling description for implementing an exemplary embodiment. It being understood that various changes may be made in the function and arrangement of elements without departing from the spirit and scope as set forth in the appended claims.

Parkinson's disease (PD) is a slow and progressive disorder and loss of dopamine producing neurons occurs over a long period of time. This suggests that a therapeutic method that can provide protection for remaining dopaminergic neurons and promote growth and restoration of other dopaminergic neurons would present a logical and valuable approach for PD treatment. Therefore, protection/restoration effects of several neurotrophic factors have been examined over the past two decades see for Unsicker “Growth factors in Parkinson's disease.” (Progress in Growth Factor Research, Vol. 5(1), pp 73-87), Lindsay “Neuron saving schemes” (Nature, Vol. 373(6512), pp 289), Connor et al “The role of neuronal growth factors in neurodegenerative disorders of the human brain” (Brain Research Reviews, Vol. 27(1), pp 1-39), and Hughes et al “Activity and injury-dependent expression of inducible transcription factors, growth factors and apoptosis-related genes within the central nervous system” (Progress in Neurobiology, Vol. 57(4), pp 421-450).

It has been proven through various preclinical studies that glial cell line-derived neurotrophic factor (GDNF) is the most effective nerve growth factor for PD treatment both in terms of restoration and protection, see for example Alexi and Gash. GDNF is a rather large regenerative molecule and belongs to the transforming growth factor beta (TGFβ) family. Due to its size it cannot pass through the human blood brain barrier (BBB) and it also becomes depraved in the body very fast. Accordingly, at present direct administration of GDNF into the brain is the only possible method, see for example Jollivet, Aoi, Popovic, and Bilang-Bleuel.

A “drug” as used herein and throughout this disclosure, refers to a material having a positive effect upon the neurotransmitter function within the brain. As such a drug may include, but not be limited to, a neurotrophic factor, a neurotransmitter, a protein, a neurotrophin, a glial cell-line derived neurotrophic factor family ligand, and a neuropoietic cytokine.

1. Prior Art:

Within the prior art there are techniques relating to growth factor intracranial delivery strategies. However, each approach faces difficulties which are outlined briefly below together with the improvements from a neurotrophic factor delivery microsystem (NEUFADEMS) according to embodiments of the invention by the inventors and how these can mitigate these disadvantages.

1A. Direct Injection or Infusion by Minipump:

Studies on animal models of PD suggest that this method is effective if the GDNF is delivered directly into the ventricular when nigrostriatal pathway is damaged, see for example Grondin et al. “Glial cell line-derived neurotrophic factor (GDNF): a drug candidate for the treatment of Parkinson's disease” (J. of Neurology, Vol. 245, pp 35-42). In this method ventricular infusion is done by using osmotic minipump. In a different study rat's nigrostriatal dopaminergic system was recovered by a single or continuous injection of GDNF in to its striatum, see for example Aoi et al. “Single or continuous injection of glial cell line-derived neurotrophic factor in the striatum induces recovery of the nigrostriatal dopaminergic system” (Neurologic al Res., Vol. 22(8), pp 832-). However, it is important to consider that GDNF is helpful only when delivered at the lesion site, see for example Kearns et al. “GDNF protection against 6-OHDA: time dependence and requirement for protein synthesis” (J. of Neuroscience, Vol. 17(18), pp 7111-).

The advantage of this administration strategy is full control over the delivered GDNF dosage. Nevertheless the main disadvantage is the high concentration of this recombinant protein at the infusion site which can damage the tissue and develop edema, see for example Gill. Also is still unclear whether single or continuous injection is more effective, see for example Kearns, as the results vary within the different trials reported to date. The inventors believe that the proposed NEUFADEMS should overcome these setbacks as the NEUFADEMS allows the dopamine concentration to be determined and then establish the infusion rate and GDNF dosage. Accordingly, it injects GDNF only when it is needed.

1B. Microsphere:

An interesting drug delivery method is using biocompatible polymer microspheres. As opposed to direct injection these biodegradable beads allow slow release of medication. This method achieved some encouraging results for cancer therapy, see for example Allison “Yttrium-90 microspheres (TheraSphere and SIR-Spheres) for the treatment of unresectable hepatocellular carcinoma” (Iss. in Emerging Health Tech., Vol. 102, pp 1). Microspheres can also be used for GDNF delivery. Studies show that implanting microspheres which contain GDNF in the striatum of PD rats improves their motor function, see for example Jollivet. The benefits of this method are the slow release of GDNF and its biocompatibility in addition to fewer side effects. On the other hand, there are some concerns regarding the non constant drug release and insufficient GDNF dosage, see for example Jollivet. Another drawback is the short distance of GDNF diffusion, see for example Salzman, which is due to the molecule binding rapidly to tissue. Accordingly the NEUFADEMS according to embodiments of the invention can rectify some of these problems by promoting personalized neurotherapy. It controls the GDNF dosage and infusion rate based on each individual patient needs as well as delivering GDNF at the exact location where it is needed.

1C. GDNF Gene Therapy:

In vivo GDNF expression by transferring recombinant viruses such as adenovirus (Ad), adeno-associated virus (AAV) and lentivirus (LV) is another growth factor delivery strategy exploited by researchers, see for example Bilang-Bleuel; Ridoux et al. “Adenoviral vectors as functional retrograde neuronal tracers” (Brain Research, Vol. 648(1), pp 171-175); Mandel et al. “Midbrain injection of recombinant adeno-associated virus encoding rat glial cell line-derived neurotrophic factor protects nigral neurons in a progressive 6-hydroxydopamine-induced degeneration model of Parkinson's disease in rats.” (Proc. of National Academy of Sciences of USA, Vol. 94(25), pp 14083); and Brizard et al. “Functional reinnervation from remaining DA terminals induced by GDNF lentivirus in a rat model of early Parkinson's disease” (Neurobiology of Disease, Vol. 21(1), pp 90-101). This method provides continuous and local GDNF production over the mentioned delivery methods which need to be refilled and microspheres that face protein instability. Experimental studies showed that injection of GDNF expressing Ad vector in rat's striatum stopped PD progression by protecting dopaminergic neurons, see Bilang-Bleuel and Ridoux. The major drawback is that a resulting immune response to Ad vectors can be quite strong, see Choi-Lundberg et al. “Dopaminergic neurons protected from degeneration by GDNF gene therapy” (Science, Vol. 275(5301), pp 838). In order to rectify this problem AAV which has low immunogenicity replaced Ad, see for example Mandel. Currently AAV viral vector is the most common method for in vivo GDNF expression.

These experimental studies suggest that gene therapy is effective only if started at the early stage of PD, see for example Bilang-Bleuel, Ridoux, Mandel and Brizard. However, unfortunately Parkinson's symptoms occur only after loss of more than 50% of dopaminergic neurons, see for example Yurek et al. “Dopamine cell replacement: Parkinson's disease” (Ann. Rev. of Neuroscience, Vol. 13(1), pp 415-440). One other major concerns of this method is lack of accurate control over gene dosing after viral injection. Another important setback is gene overexpression which may modify cellular functionality, see Jakobsson et al. “Evidence for disease regulated transgene expression in the brain with use of lentiviral vectors” (J. Neuroscience Research, Vol. 84(1), pp 58-67). The risk of tumor formation due to accidental mutagenesis also adds to the complexity of this method, see Hacein-Bey-Abina and Li.

To overcome the mentioned obstacles, ex vivo gene therapy has been developed and achieved encouraging results in some experimental studies, see for example Park; Akerud et al. “Neuroprotection through delivery of glial cell line-derived neurotrophic factor by neural stem cells in a mouse model of Parkinson's disease” (J. Neuroscience, Vol. 21(20), 8108); and Cunningham et al. “Astrocyte delivery of glial cell line-derived neurotrophic factor in a mouse model of Parkinson's disease” (Experimental Neurology, Vol. 174(2), pp 230-242). In this technique GDNF expressing cells are engineered and encapsulated by a biocompatible material prior to injection. But still this strategy is beneficial only when PD is in its very early stages. In addition it is still unknown if long term GDNF delivery is beneficial, see Nutt et al. “Randomized, double-blind trial of glial cell line-derived neurotrophic factor (GDNF) in PD” (Neurology, Vol. 60(1), pp 69) and Zhang et al. “Dose response to intraventricular glial cell line-derived neurotrophic factor administration in Parkinsonian monkeys” (J. of Pharm. & Exp. Therapeutics, Vol. 282(3), 1396). These limitations promote the need for a microsystem than can act as normal healthy cells or organs. The microsystem can intelligently decide the proper dosage and infusion rate of GNDF based on real time data collected from the local environment.

2. Neurotransmitter Sensing:

In order to provide a NEUFADEMS having controlled dosage determined in dependence upon the patient's needs an initial element is that of designing a chemical sensor, capable of measuring micromolar dopamine concentrations in a format compatible with the NEUFADEMS. Previous studies suggest that electrochemical sensors are suitable for neurotransmitter sensing, see for example Murari et al. “Integrated potentiostat for neurotransmitter sensing” (Engineering in Medicine and Biology Magazine, IEEE 24(6), pp 23-29); Zhang et al. “Electrochemical array microsystem with integrated potentiostat” (IEEE Conference Sensors 2005, 4pp.); Martin et al. “A low-voltage, chemical sensor interface for systems-on-chip: the fully-differential potentiostat” (Proc. IEEE Circuits and Systems ISCAS 2004); and Poustinchi et al. “Low power noise immune circuit for implantable CMOS neurochemical sensor applied in neural prosthetics” (Proc. 5th Intnl. IEEE EMBS Conference on Neural Engineering, Paper SaE1.2). Electrochemical sensors are the largest and the most developed group of chemical sensors, see for example Janata “Principles of chemical sensors” (Springer Verlag ISBN 978-0-387-69930-1).

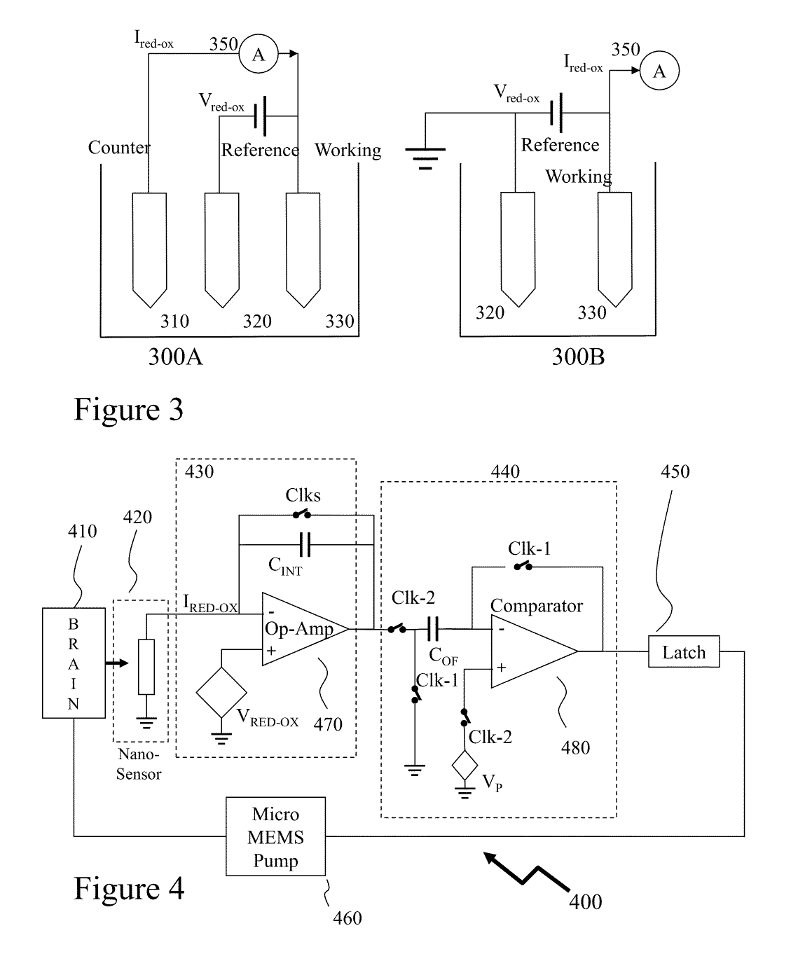

Every neurotransmitter is associated with certain voltage, see for example Robinson et al. “Detecting subsecond dopamine release with fast-scan cyclic voltammetry in vivo” (Clinical Chemistry, Vol. 49(10), 1763). To measure neurochemical concentration, this voltage is applied between the working and reference electrode. The potential difference generates a reduction-oxidation (red-ox) current which is proportional to the neurotransmitter concentration, see for example Janata, as depicted in FIG. 3 by second electro-analysis configuration 300B. This electrode configuration faces two disadvantages: first the reference electrode may become polarized if its size is 100 times smaller than working electrode, as reported by Madou et al in “Chemical sensing with solid state devices” (Academic Press ISBN 978-0-124649651); second is the material consumption due to the current in reference electrode, see Madou. To rectify these draw backs, a second 3 electrode configuration was developed as depicted by first electro-analysis configuration 300A. In this case, a third auxiliary electrode (or counter electrode) is used for current injection purposes, whilst the reference electrode has true well-defined reference potential, see for example Eggins “Chemical Sensors and Biosensors” (Wiley); Madou; and Gopel “Solid State Chemical Sensors” (J. Phys. E. Sci. Instr., Vol. 20, 1127).

There are several electrochemical techniques to measure extracellular concentration of neurotransmitters, including but not limited to, microdialysis, constant-potential amperometry, fast-scan cyclic voltammetry, high speed chronoamperometry and differential normal-pulse voltammetry, see for example Robinson. It would be beneficial for a NEUFADEMS to possess high sensitivity, high chemical selectivity, and fast temporal resolution.

However, considering the prior art techniques then although a high degree of chemical selectivity and sensitivity can be achieved with microdialysis, the method has very low temporal resolution and due to its large size is not suitable for implantable sensors. In contrast, amperometry has very low selectivity but a very high temporal resolution. Selectivity can be improved by using biological filters and coating the electrodes with Nafion, see for example Gerhardt et al. “Nafion-coated electrodes with high selectivity for CNS electrochemistry” (Brain Research, Vol. 290(2), pp 390-395). However, this process significantly decreases the life time of the electrode, see for example Fry et al. “Electroenzymatic synthesis (regeneration of nadh coenzyme): Use of nafion ion exchange films for immobilization of enzyme and redox mediator” (Tetrahedron Lett., Vol. 35(31), pp 5607-5610). Fast-scan cyclic voltammetry possesses good chemical selectivity while maintaining subsecond temporal resolution, see Robinson. Fast-scan cyclic voltammograms are repeated every 100 ms, thus changes in chemical concentration can be monitored on a sub-second time scale, see Robinson. These characteristics make fast-scan cyclic voltammetry suitable for detecting phasic neurotransmitter changes in behaving animals. Accordingly, the inventors have combined amperometry and fast-scan cyclic voltammetry to create a new dopamine sensor that takes advantage of both methods. Using both techniques at the same time results in a sensor with a high chemical selectivity while having high temporal resolution which as noted above is beneficial for a NEUFADEMS.

Within the remaining description of embodiments of the invention the results presented for the NEUFADEMS Nafion coated carbon fiber electrodes were employed, see for example Momma et al. “Electrochemical modification of active carbon fiber electrode and its application to double-layer capacitor” (J. Power Sources, Vol. 60(2), pp 249-253). Potentially these electrodes may not prove suitable for long term implantation. Accordingly, the inventors believe that novel dopamine specific nanowire sensors may rectify this limitation.

3. Neurotransmitter Sensing Circuit Architecture:

To measure dopamine concentration and control GDNF administration within a NEUFADEMS according to embodiments of the invention a low power, low noise CMOS circuit would be beneficial. Referring to FIG. 4 there is depicted circuit schematic 400 according to an embodiment of the invention. The NEUFADEMS circuitry consists of two major components. The first is a current conveyor that establishes the V_{RED-OX} voltage between the sensor electrodes within the nano-sensor 420 implanted into the patients brain 410. Then the integrating capacitor 490 collects the corresponding current which is proportional to dopamine concentration. The second component is comparator 440 which compares the recorded voltage with a reference voltage, V_{P}. V_{P} is a voltage threshold established as presenting a minimum acceptable dopamine concentration within the nigrastriatal pathway of the patient. If the recorded voltage is less than V_{P}, it sends an “ON” signal to micro MEMS pump 460 to inject required GDNF otherwise the micro MEMS pump 460 is turned off.

It would be evident for one skilled in the art that it would be beneficial for any implantable circuit to operate with minimum power consumption to minimize heating effects for example and extend lifetime of such a NEUFADEMS from a battery to support mobility of the patient. Accordingly, this sensing and controlling circuit depicted in circuit schematic 400 was designed and implemented with standard 0.18 μm CMOS processes resulting in a total power consumption of 921 nW whilst the sensing circuit still maintains approximately 2 kHz bandwidth.

3A. Low Power Noise Immune Current Conveyor:

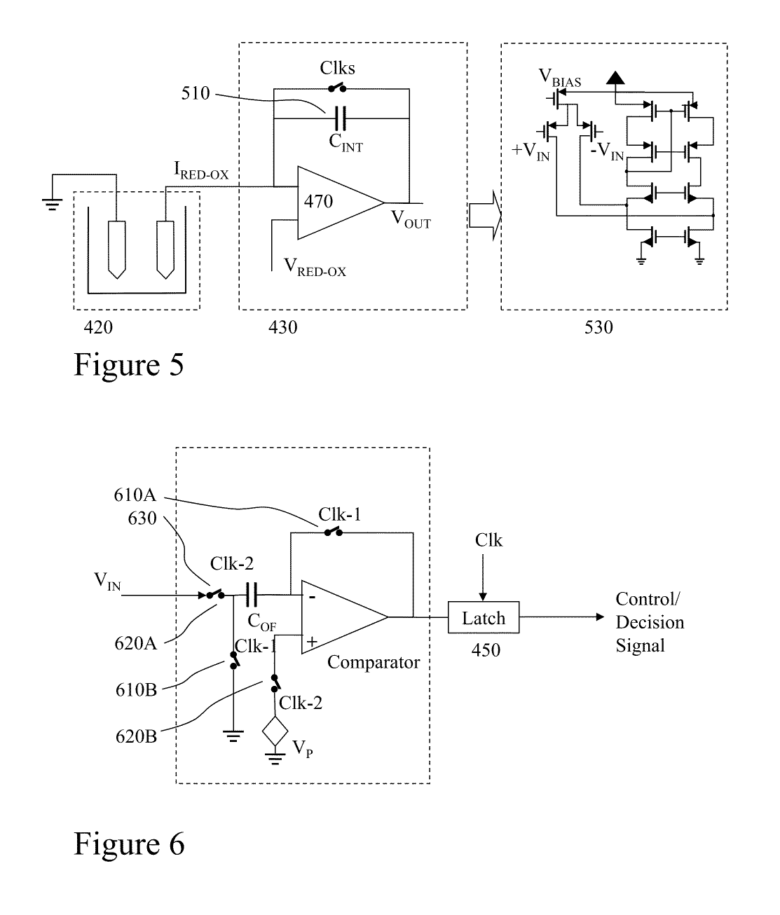

To measure the electrochemical current, the red-ox potential is applied between a working and a reference electrode. The current conveyor 430 converts the resulting red-ox current, which is in the pico-amp to nano-amp range, to voltage. The central element of the current conveyor 430 is the operational amplifier (op-amp) 470. Instead of using a front end amplifier with high power consumption a wide swing folded cascade amplifier, such as depicted by amplifier 530 in FIG. 5 is used for its high gain and stability, see for example Mandal et al “Self-biasing of folded cascade CMOS op-amps” (Intnl. J. Elect., Vol. 87(7), pp 795-808). Such folded cascade amplifiers minimize power dissipation as the resulting operational amplifier 470 is accordingly designed to operate in the sub-threshold region. Amplifier 530 whilst providing low power consumption also provides high gain and low bandwidth. The inventors have demonstrated that the resulting current conveyor 430 is not only low power but also high noise immunity, see Poustinchi and Musallam “Low power noise immune circuit for implantable CMOS neurochemical sensor applied in neural prosthetics” (Proc. 5th Intnl. EMBS Conf. on Neural Engineering, 2011). Within the design for the NEUFADEMS the power consumption is further reduced by decreasing the unity gain bandwidth.

Accordingly, the potential applied to the neurochemical sensor, V_{RED-OX}, generates an effective current, I_{RED-OX}, due to the resistance, R_{SENSOR}, between the reference electrode and working electrode. Accordingly, this current I_{RED-OX} is proportional to neurochemical concentration at the sensor and accumulates charge on the capacitor C_{INT} 510 over a predetermined over integration period, T_{INT}. The output voltage of the current conveyor 430 comprising amplifier 530 with the capacitor C_{INT} 510 is calculated by Equation (1) below. In addition since integration is an averaging operation the current conveyor 430 has high noise immunity. Implemented within 0.18 μm CMOS the amplifier 530 consumes only 0.47 μW which is amongst the lowest reported to date, see for example Mandal and Yao et al “A 1V 140 W 88 dB audio sigma-delta modulator in 90 nm CMOS” (IEEE J. Solid-State Circuits, Vol. 39(11), pp 1809-1818). The specification for the amplifier 530 are presented below in Table 1 together with similar prior art amplifiers.

$${V}_{OUT} = \frac{1}{{C}_{INT} \times {R}_{SENSOR}} {\int }_{0}^{{T}_{INT}} {V}_{RED \text{-} OX} d t \qquad ( 1 )$$

TABLE 1
Amplifier Specifications and Comparison

| Specification | Mandal | Yao | Inventors |
|---|---|---|---|
| Architecture | Class AB | Telescopic | Folded Cascade |
| Technology (μm) | 0.09 | 0.18 | 0.18 |
| DC Gain (dB) | 50 | 79 | 65.1 |
| Unity Gain Bandwidth | 57 | 8.5 | 4.75 |
| (MHz) | | | |
| Phase Margin (deg) | 57 | 78 | 65 |
| Supply Voltage (V) | 1 | 0.925 | 1 |
| Output Swing (V) | [−0.2, +0.2] | [−0.2, +0.2] | [−0.45, +0.43] |
| Power (μW) | 80 | 4.6 | 0.47 |

3B. Comparator with Offset Cancelation:

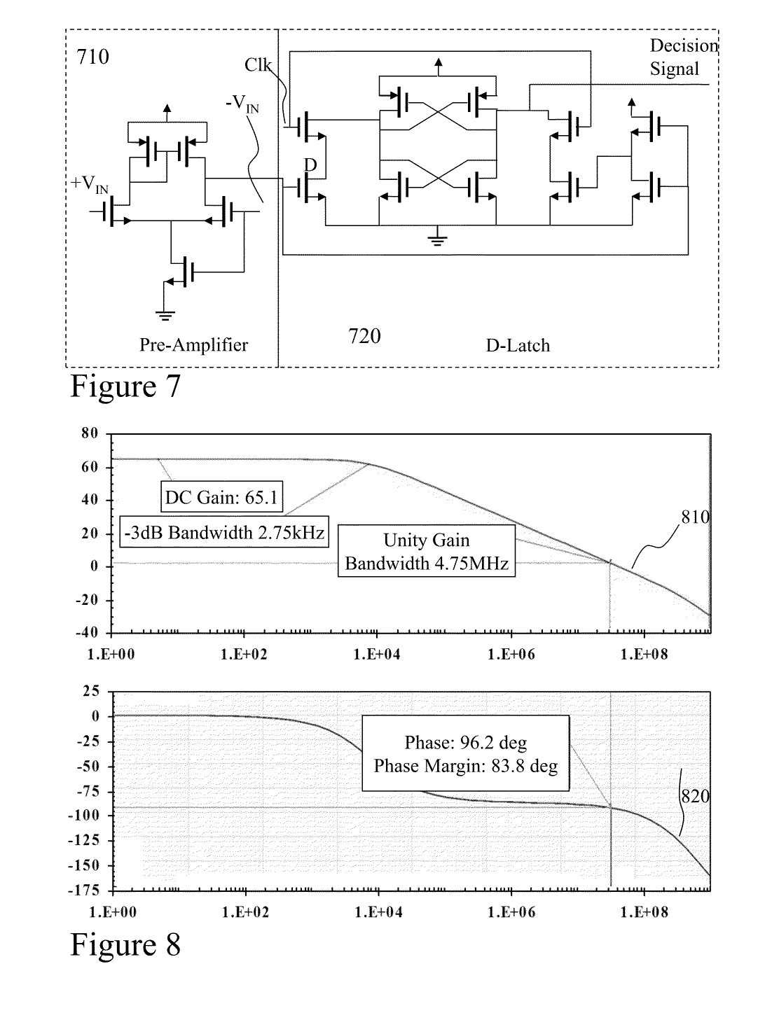

To compare the measured dopamine concentration with its nominal value in substantia nigra, a low power comparator 440 was designed followed by a digital latch 450 as depicted in FIG. 4. In order to improve the performance of comparator 440 an auto-zero offset cancellation technique was exploited, see for example Enz et al “Circuit techniques for reducing the effects of op-amp imperfections: auto zeroing, correlated double sampling, and chopper stabilization” (Proc. IEEE, Vol. 84(11), pp 1584-1614). Referring to FIG. 6 the comparator 440 is depicted in isolation from the remainder of the circuit. In a first phase first and second Clk-1s 610A and 610B respectively are “ON” and capacitor 630, C_{OF}, stores an offset voltage for a pre-amplifier stage within the comparator block 480 within the comparator 440. Such a pre-amplifier stage being depicted by pre-amplifier 710 in FIG. 7 for example. In a second phase first and second Clk-2s 620A and 620B are “ON” such that this offset voltage is eliminated by its being subtracted from V_{IN}. Equations (2) and (3) illustrate the cancelation technique where A is open loop gain of the pre-amplifier 710 within the pre-amplifier stage of the comparator block 480 within the comparator 440.

Phase 1 V₋=V_{OFFSET} (2)

Phase 2 V_{OUT}A×(V₊−V₋) (3A)

V_{OUT}=A×(V_{P}+V_{OFFSET}−V_{IN}−V_{OFFSET}) (3B)

V_{OUT}A×(V_{P}−V_{IN}) (3C)

There are several circuit topologies for comparators and the one depicted and employed within embodiments of the invention is a so-called latched comparator wherein the comparator 440, employing a low gain pre-amplifier (e.g. 25 dB), is followed by a D-type Latch depicted by Latch 450 within FIG. 6. The op-amp based comparator 440 minimizes the kick-back noise whilst the latch 450 acts as positive feedback and its output swings between “low and “high” levels according to the input logic thresholds of the micro MEMS pump 460 within the NEUFADEMS as depicted by circuit schematic 400 in FIG. 4. For example, these levels are set to nominal 0V and 1.8V such that the D-latch swings between these levels.

The D-latch stores comparator's state until the next comparison. D-latch 720 within FIG. 7 presents one exemplary embodiment of a D-latch. Accordingly, when V_{IN}, being the output of the current conveyor 430 and corresponding to a dopamine concentration, is less than V_{P}, then the comparator 440 sends an “ON” signal to an actuator within the micro MEMS pump 460 to inject GDNF. The NEUFADEMS continually compares the dopamine concentration determined from the sensor with its nominal set-point value. When it reaches the normal value, i.e. V_{IN}≦V_{P} then the comparator 440 sends an “OFF” signal to micro MEMS pump 460, stopping the GDNF injection. By applying low power design techniques the inventors have designed and demonstrated very low power comparators 440 for such NEUFADEMS with only 451 nW power dissipation.

3C. Results and Comments on Neurotransmitter Sensing Circuit Architecture:

In order to determine the DC gain, phase margin, and 3 dB frequency of the neurotransmitting circuit elements AC analysis of the op-amp 470, which is used in current conveyor, is necessary. Accordingly, a differential sinusoidal signal with 0.5 volt amplitude and 0 and 180 degree phase was applied to each input terminals and the Bode plot generated from output signal. Referring to FIG. 8 the gain and phase measurements for a sensing circuit according to an embodiment of the invention are shown in FIG. 8 as a function applied drive frequency from 1 Hz to 100 MHz showing 3 dB gain bandwidth of approximately 2.75 kHz and unity gain bandwidth of approximately 4.75 MHz where the phase margin is approximately 84 degrees.

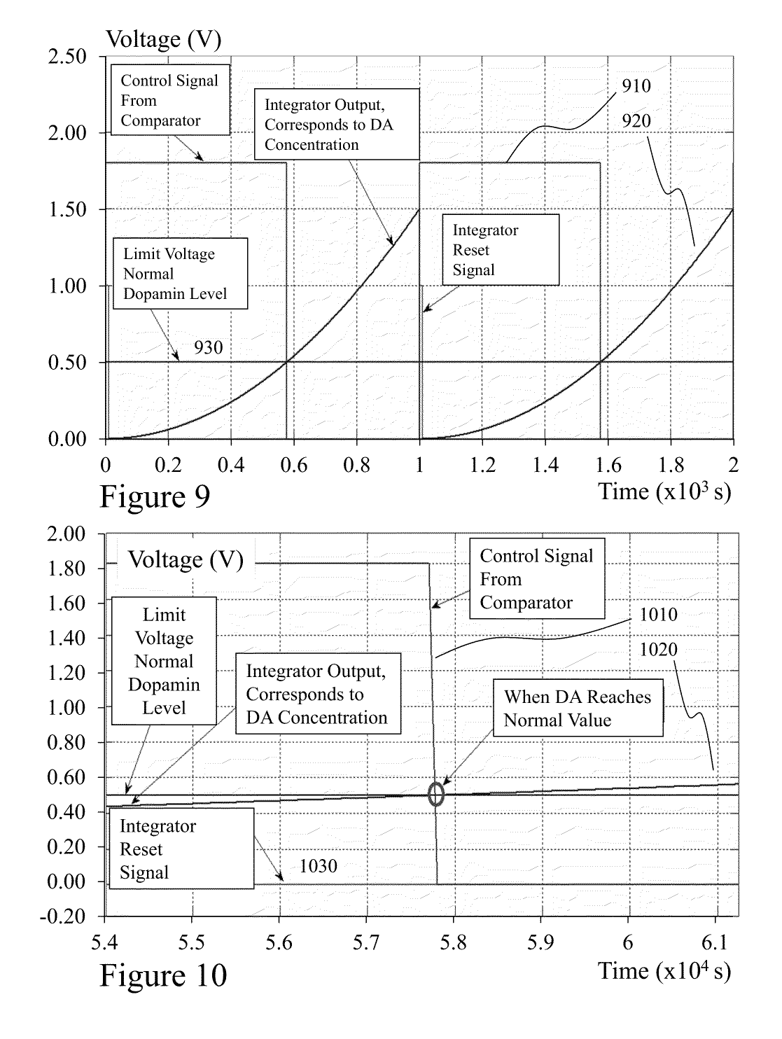

The NEUFADEMS electrical functionality was evaluated using transient analysis obtained by applying a sawtooth current with 24 nA peak and 1 ms period to the NEUFADEMS. This signal resembles dopamine concentration as reported by Michael et al “Electrochemical methods for neuroscience” (CRC). Analysis indicates that the normal dopamine concentration in a healthy rat generates approximately 8 nA current. Based upon choosing the integration period to be 1 mS and integration capacitor to have a value of 16 pF this implies a 0.5V voltage would be generated at the output of current conveyor. Setting 0.5V to the reference voltage implies that if the measured voltage is less than 0.5V, dopamine concentration is less than the normal value, such that the comparator sends an “ON” signal to the micro MEMS pump to inject GDNF. These transient measurements are presented in FIG. 9.

The integration period and capacitor value were selected only for evaluation and electrical validation of the NEUFADEMS circuit elements. Accordingly these values are subject to variation based on experimental results of GDNF within humans and the variations of GDNF dynamics with factors including but not limited to characteristics of the patient, region of the brain and long-term dynamics of neurotrophic factor injection delivery. Referring to FIG. 10 it can be seen that when the dopamine concentration reaches its normal value the comparator turns the actuator “OFF” and stops GDNF injection. In addition in order to avoid integration saturation, a reset signal is activated every one millisecond. Optionally this reset signal may be triggered with different time bases as well as based upon other measurements and/or characteristics.

4: Digitization of Neurotransmitter Sensor Output:

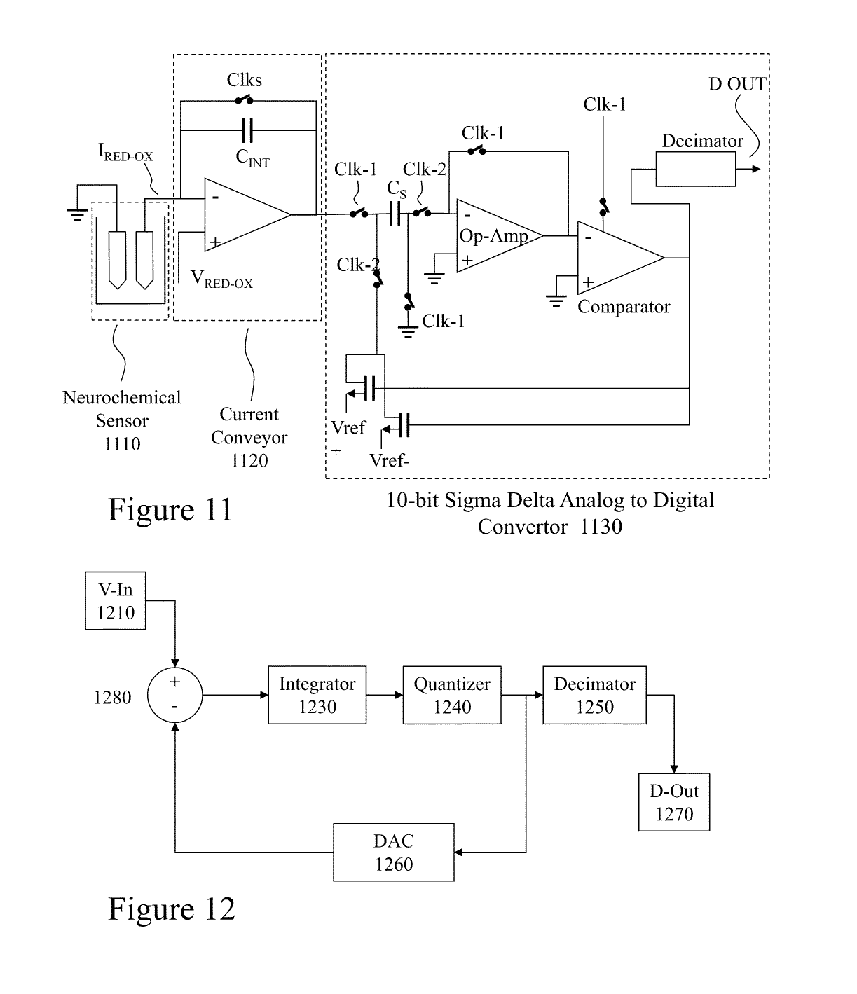

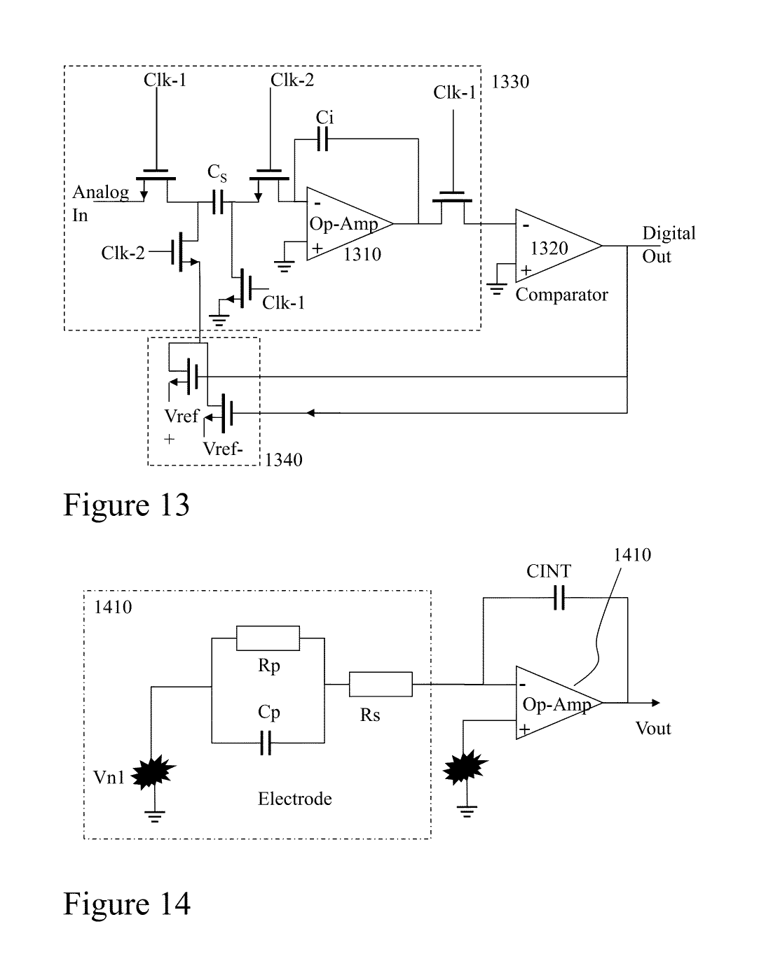

In the preceding sections a NEUFADEMS employing a CMOS potentiostat in conjunction with CMOS current conveyor, comparator, and latch was presented to provide a low power feedback loop for controlling a MEMS pump for the delivery of GDNF. Such a NEUFADEMS operates with “digital” control of the MEMS pump in that the output from the CMOS current conveyor, comparator, and latch was either logic “0” or logic “1” thereby turning the pump “OFF” and “ON”. In other scenarios it would be beneficial for the output of a neurotransmitter sensor to be digitized thereby providing a measurement of the neurotransmitter to a microprocessor or other digital controller wherein the data may be stored or employed in establishing delivery at multiple levels. Such a digital neurotransmitter sensing circuit is depicted in FIG. 11 comprising a neurochemical sensor 1110 such as described above in respect of FIG. 3, current conveyor 1120 such as described above in respect of FIGS. 5, and 10-bit Delta-Sigma ADC 1130.

As discussed above an integrated potentiostat was reported by Murari et al. This potentiostat employed delta sigma analog-to-digital converters (ADCs) for each sensor channel instead of using off-chip ADCs or a single ADC for several channels with multiplexing. Although this design reduced power consumption and noise compared with such commercial off-chip ADCs the ADC components in the Murari design still required high power. However, for brain implant circuits low power dissipation is vital and impacts not only patient comfort but patient quality of life through generating less heat but establishing mobile device lifetime from battery based power sources and allowing smaller energy sources.

4A: Amplifier Specifications and Comparison:

A 10-bit first order Delta-Sigma Analog-to-Digital Converter (ADC) was designed to convert the current conveyor's output voltage into a digital code. A Delta-Sigma ADC was chosen for its high resolution, low power and small area and implemented with 10-bit code conversion compared to the single-bit Delta-Sigma ADC of Murari. As the chemical reactions being monitored with respect to neurotransmitters and other brain processes for neurological disorders are slow, typically millisecond to second timescales the requirement for a high speed ADC is absent for these applications. Delta Sigma ADCs owes their performance to oversampling and noise shaping wherein quantization noise is pushed out of the band of interest.

Referring to FIG. 12 there is depicted a functional schematic of a Delta-Sigma ADC according to embodiments of the invention wherein the received voltage output from the current conveyor 1210 is coupled to a Combiner 1280 the output of which is coupled to an Integrator 1230 and Quantizer 1240 in the forward path wherein the Quantizer 1240 output is coupled to a Digital-to-Analog Converter (DAC) 1260 in a feedback path to the Combiner 1280 and fed forward to a Decimator 1250 which generates the digital output 1270.

Referring to FIG. 5 the Integrator 1230 is depicted comprising a dual-stage operational amplifier (op-amp) 1310 in conjunction with switch-capacitor circuit 1330. Clk1 and Clk2 are non-overlapping clocks controlling application of the feedback and input signals to the dual-stage op-amp 1310 as well as gating the output of the dual-stage op-amp 1310 to the comparator 1320 which acts as the Quantizer 1240. In order to minimize the kick-back noise a pre-amplifier followed by a D-Latch were employed to form comparator 1320. In order to reduce the overall die area, which is important for implantable circuits, a simple two switch circuit 1340 was employed to provide the DAC 1260 in the feedback path which is fed by the output of the comparator 1320. A primary ADC design goal was to minimize the power consumption while meeting required specifications leading to a reduction in sampling frequency and low power biasing.

Fabricated 10-bit first order Delta-Sigma ADCs in 0.18 μm CMOS demonstrated power dissipation of 120 μW which is lower than similar designs, see for example Keogh “Low-Power Multi-Bit-Modulator Design for Portable Audio Application” (Royal Institute of Technology, M.Sc Thesis, Stockholm, March 2005); Agah et al. “A high-resolution low-power oversampling ADC with extended-range for bio-sensor arrays” (IEEE Symp. VLSI Circuits 2007, pp 244-24-5); and Lee et al “A low-voltage and low-power adaptive switched-current sigma-delta ADC for bio-acquisition microsystems” (IEEE Trans. Circuits and Systems I, Vol. 53(12), pp 2628-2636). The measured ADC bandwidth was approximately 1.5 kHz while sampling at 384 kHz with 66.1 dB Signal-to-Noise Ratio (SNR) which is equivalent to 10-bit resolution as determined by Equation 4. The Oversampling Ratio (OSR) was 128, where Equation (5) demonstrates the relationship between bandwidth, sampling frequency and oversampling ratio. Table 2 presents the measured performance of the 10-bit first order Sigma-Delta ADC according to an embodiment of the invention with results from Keogh, Agah, and Lee.

$$\begin{matrix}BitR = \frac{S N R ( dB ) - 1.76}{6.02} & ( 4 ) \\ B W = \frac{{F}_{SAMPLING}}{2 \times O S R} & ( 5 )\end{matrix}$$

where BitR is the Bit Resolution, BW is the bandwidth, and F_{SAMPLING} the sampling frequency.

TABLE 2
Sigma Delta ADC Specifications and Comparison

| Specification | Keogh | Agah | Lee | Inventors |
|---|---|---|---|---|
| Technology (μm) | 0.18 | 0.18 | 0.18 | 0.18 |
| SNR (dB)/#-bit | 85.76/13 | 60/9 | 67.8/10 | 66.1/10 |
| Bandwidth (kHz) | 50 | 5 | 4 | 1.5 |
| Supply Voltage (V) | 1.8 | 0.8 | 1.8 | 1 |
| Power (μW) | 38000 | 180 | 400 | 121 |

4B: Noise and Power Analysis:

The major components of the NEUFADEMS on the neurotransmitter sensor and digitization, the current conveyor and ADC respectively, have been designed to have minimum power dissipation. The total current pulled from the power supply by the designed microsystem is approximately 67 μA. Accordingly, using Equation (6) the total power consumption was calculated as approximately 121 μW.

Power=V×I_{TOTAL}=1.8×67.13=120.83 μW (6)

Referring to FIG. 14 there is shown a simplified circuit for the microsystem's front end in addition to the electrode model and noise sources. There are two possible noise sources, denoted by Vₙ₁ and Vₙ₂ respectively wherein Vₙ₁ represents the noise of the sensing electrode and Vₙ₂ represents the input referred noise of the amplifier. Since this circuit operates in low frequency, the series resistance of the electrode is negligible. Accordingly, the input referred current noise is formulated as per Equation (7) below. Accordingly it is evident that in order to minimize the input referred current noise and improve sensors selectivity Vₙ₁ and Vₙ₂ should both be reduced. Differential pair and bias transistors in the folded cascade transistor have maximum contribution to input referred noise of the amplifier. To minimize their effect they were designed to operate in strong inversion. Total current input referred noise of this NEUFADEMS sensing front-end over the bandwidth of interest is approximately 0.6 fA (femtoamp) which is three orders of magnitude lower than the device selectivity which is picoamperes (pA). Additionally, the integration within Equation (1) represents an averaging operation and provides significant noise immunity. It would be evident to one skilled in the art that the larger the time-constant of the integration the higher the noise rejection capability of the circuit.

$${I}_{n}^{2} = {\vert j ω {C}_{p} + \frac{1}{{R}_{p}} \vert}^{2} \times ( {V}_{n 1}^{2} + {V}_{n 2}^{2} ) \qquad ( 7 )$$

4C: Simulation Results:

The 10-bit first order Sigma-Delta ADC was tested by computing the Fast Fourier Transform (FFT) of the output to calculate the power and Signal-to-Noise-Ratio (SNR). The Power Spectral Density (PSD) and SNR were calculated using Equations (8) and (9) respectively.

$$\begin{matrix}P S D = 10 log ( power ) & ( 8 ) \\ S N R = \frac{{Power}_{OUT \text{-} SignalBin}}{{\sum }_{j}^{N} {Power}_{OUT \text{-} JthBin}} & ( 9 )\end{matrix}$$

where j is the first bin outside of the bandwidth and N is total number of samples.

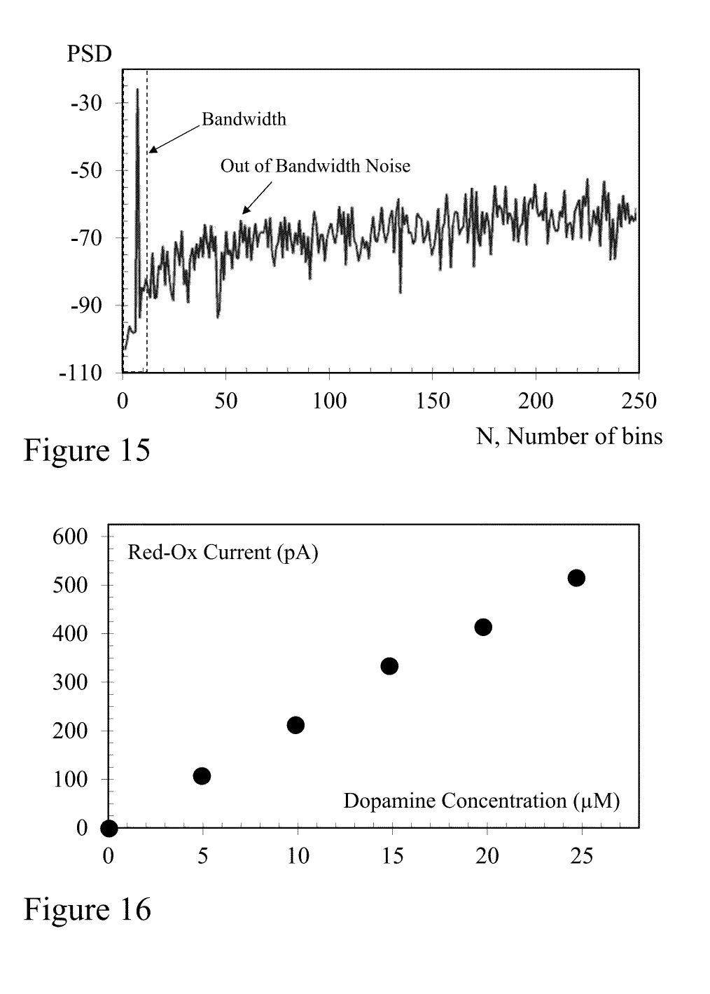

Referring to FIG. 15 there is presented PSD of the 10-bit first order Sigma-Delta ADC. The total number of samples is 1024 and Over Sampling Ratio (OSR) is 128. The input signal frequency is 1.125 kHz with 0.15V amplitude peak to peak. The calculated SNR is 66.1 dB which is equivalent to 10-bit.

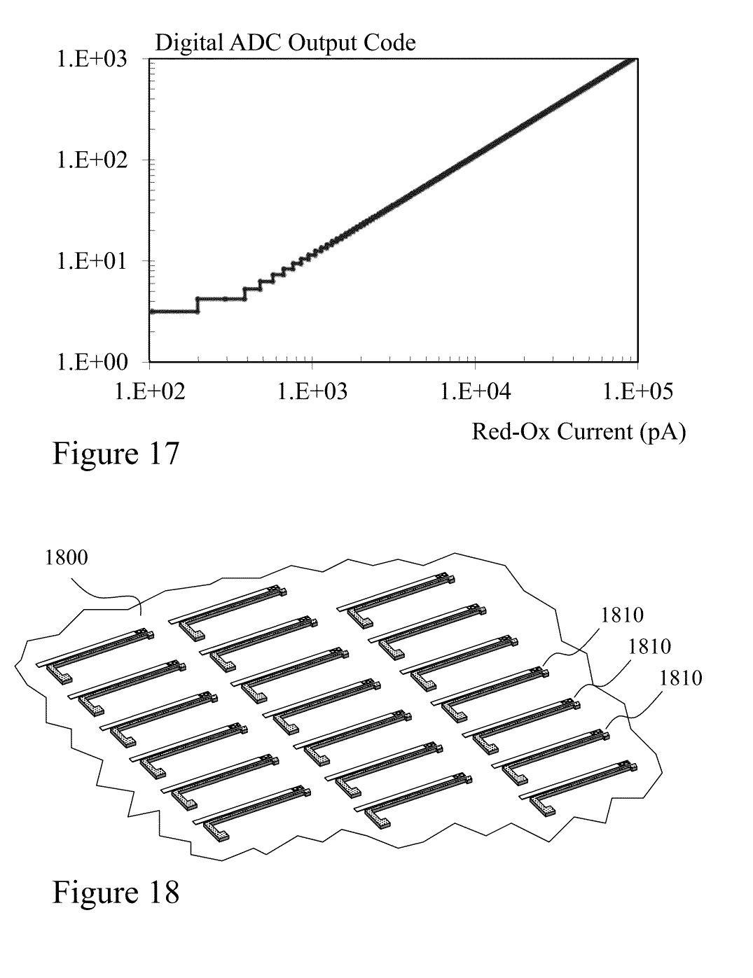

FIG. 16 depicts results obtained from measurements using a VersaSTAT 4 potentiostat from Princeton Applied Research which is a laboratory test instrument. FIG. 16 depicts the measured red-ox current in response to addition of 5 μM (micromolar) Dopamine thereby showing the red-ox current with increasing Dopamine concentration wherein it is clear that an approximate slope of 20 pA/μM. Accordingly, to test the neurochemical micro sensor the input current was swept from 5 μM to 5,000 μM. FIG. 17 depicts the resulting conversion of the red-ox current to 10-bit digital code.

5. Neufadems.

Within the preceding sections 3 and 4 neurotransmitter sensors together with decision and digitization circuits have been outlined according to embodiments of the invention which provide very low drive power when implemented in 0.18 μm CMOS providing for monolithic integration of these electronic circuits with other elements including, but not limited to, MEMS based pumps, microfluidic channels and reservoirs, optical sensors, electrical stimulation circuit, control electronics, digital signal processing circuits, digital memory, and a microprocessor.

Accordingly using standard 0.18 μm CMOS processes, rather than leading edge 55 nm, 65 nm, and 90 nm processes, low cost manufacturing on wafers is currently possible up to 300 mm (12 inch). Accordingly manufacturing processes may be performed prior to separation of the tapered probes such that all manufacturing processes are performed on arrays of devices such as shown in FIG. 18 wherein the probes 1810 are formed in array across the substrate 1800 It would be evident to one skilled in the art that multiple process flows may be implemented without departing from the scope of the invention.

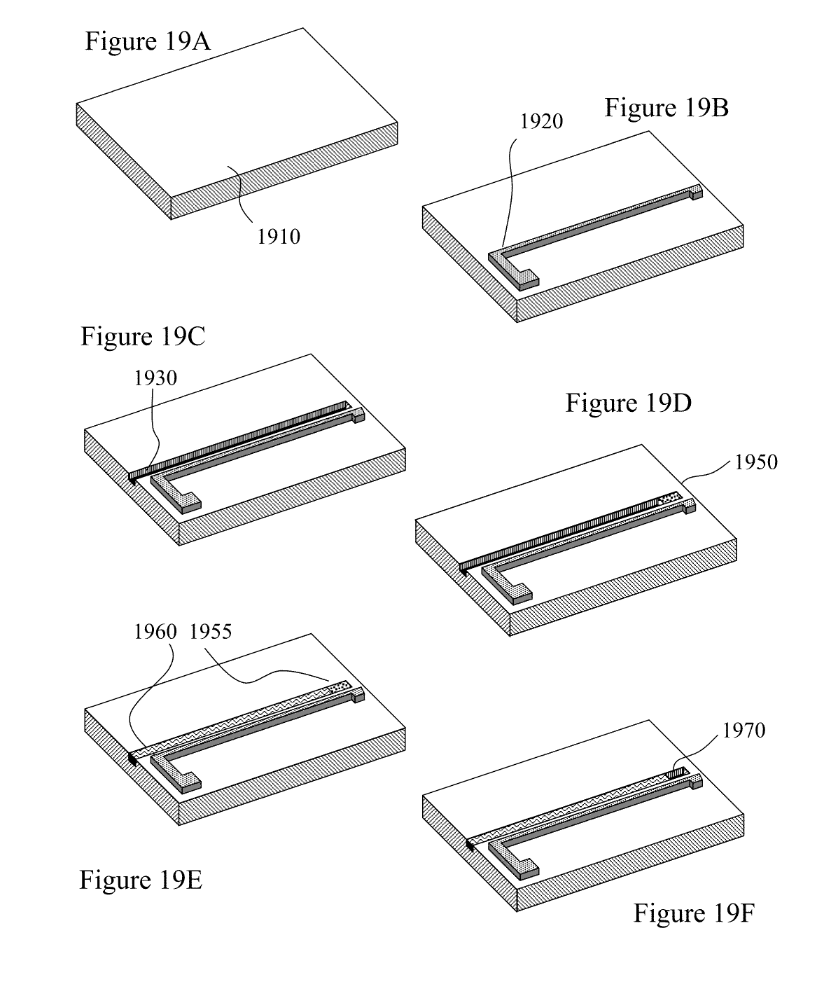

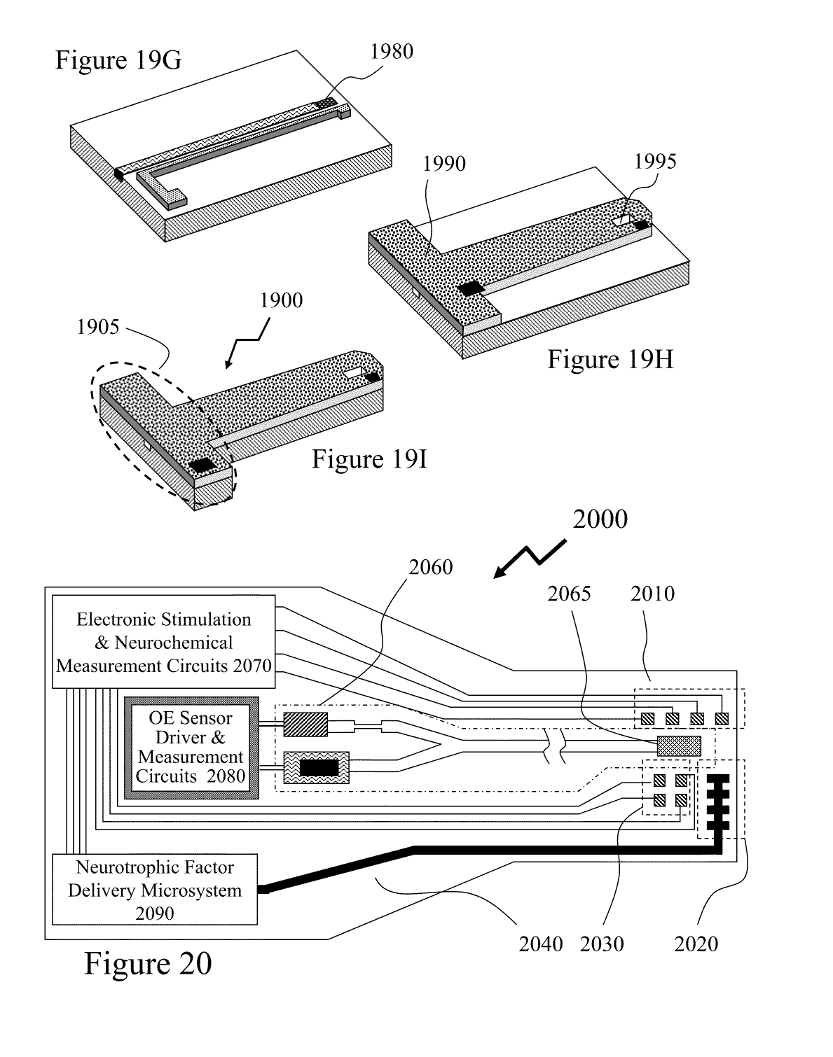

Referring to FIG. 19A through to 19I there is shown an exemplary process flow for the manufacturing an electrical interconnection and microfluidic channel according to an embodiment of the invention wherein the electrical interconnection and microfluidic channel comprise portions of a brain probe comprising a neurotrophic factor delivery microsystem in conjunction with an optoelectronic sensor and electronic stimulation and neurochemical measurement circuits. The process beginning in FIG. 19A with the provisioning of a 100 microns thick double side polished silicon wafer 1910. Within this embodiment of the invention the silicon wafer is boron doped with a resistivity of 20 ohm-cm and having a <100> orientation. Next in FIG. 19B a 20 nm thin layer of titanium is deposited by sputtering. This layer serves as an adhesion layer between the silicon wafer 1910 and the subsequent 100 nm thick gold layer deposited on the titanium also by sputtering forming electrode metallization 1920. These metal layers are patterned by photolithography and etching to form the recording sites, interconnections and bond pads.

Subsequently a resist layer is patterned with photolithography and the exposed silicon is etched using an anisotropic XeF2 or DRIE system to form for example a 100 μm wide rectangular cavity 1930 of depth 20 μm. This being shown in FIG. 19C. Next in FIG. 19D a second photolithographically patterned resist layer is used to protect the region 1950 within the rectangular cavity 1930 which will subsequently contain a porous neurotrophic dispensing site within the probe.

Using a third photolithography stage the remainder of the rectangular cavity 1930 is filled with a sacrificial material 1960 to protect the microfluidic channel as shown in FIG. 19E. Next the second photolithographically patterned resist layer is removed leaving behind a polymeric filled channel with a cavity 1970 as shown in FIG. 19F. Then using a fourth photolithographic process the cavity 1970 is filled within an appropriate porous material 1980 as shown in FIG. 19G such as for example a xerogel. Finally the probe is coated with Parylene™ C 1990, a chemical vapor deposition compatible poly-xylylene polymer with chlorine, and patterned in order to expose the porous material 1980 through opening 1995 as shown in FIG. 19H. Next as shown in FIG. 19I the structure is patterned by etching the exposed silicon completely by XeF2 or DRIE systems which result in a tapered probe 1900 as shown in FIG. 19I with wide base carrier area 1905. If the tapered probe 1900 is formed in a row then the individual tapered probes 1900 may also be separated by dicing or cleaving. Next the sacrificial material 1960 is removed to provide the empty microfluidic channel. Alternatively the sacrificial material 1960 may be removed prior to providing the coating layer to the structure. Optionally the porous material 1980 may be provided through a direct-dispense technique either to implement a modified process flow or to allow use of a material otherwise not compatible with the semiconductor processing techniques.

As described within FIG. 19A through 19I the microfluidic channel and electrical interconnections are described as being formed on the same side of the silicon wafer 1910 which is an ultra-thin wafer. Alternatively the silicon wafer 1910 may be a thicker wafer which is processed either at the end of the process flow or at an intermediate processing point using chemical-mechanical planarization to the desired thickness. It would also be possible to employ silicon crack propagation as reported by IMEC (http://www.sciencedaily.com/releases/2008/07/080714144222.htm) wherein a full thickness silicon wafer once processed has a crack induced approximately 30 microns deep into the structure and is propagated across the wafer. Similarly epitaxial lift off of epitaxially grown silicon on porous silicon has been demonstrated for removal of large area ultra-thin silicon (http://www.imec.be/wwwinter/mediacenter/en/SR2003/scientific_results/research_imec/2_{—}4_ph oto/2_{—}4_{—}2/2_{—}4_{—}2_{—}1.html).

Now referring to FIG. 20 there is depicted an exemplary probe configuration 2000 comprising a neurotrophic factor delivery microsystem according to an embodiment of the invention in conjunction with an optoelectronic sensor and electronic stimulation and neurochemical measurement circuits. As depicted the probe configuration 2000 comprises electrical stimulation sites 2010, neurotrophic dispensing site 2020, neurotransmitter sensor site 2030, and optical sensor 2065. The electrical stimulation sites 2010 are coupled to Electronic Stimulation & Neurochemical Measurement Circuits 2070 which are also connected to Neurotrophic Factor Delivery Microsystem 2090 such that a micro MEMS pump controls delivery of the neurotrophic factor via fluidic microchannel 2040 to the neurotransmitter sensor site 2030. Optical sensor 2065 forms part of opto-electronic sensing circuit 2060 which is connected to Opto-Electronic Sensor Driver & Measurement Circuits 2080. Accordingly embodiments of the invention providing a NEUFADEMS form part of the probe configuration 2000 together with the electrical stimulation sites 2010, opto-electronic sensing circuit 2060 and Opto-Electronic Sensor Driver & Measurement Circuits 2080.

Each of the electronic circuits may couple to electrical connections, not shown for clarity, such that the probe configuration 2000 forms part of a large device managing or assessing neurological issues for the patient as well as providing electrical power such as for example via a battery. Additionally, an inlet may be provided on the edge of the probe configuration 2000 coupling to the micro MEMS pump and fluidic microchannel 2040 such that the neurotrophic dispensing site 2020 is coupled to a neurotrophic factor reservoir.

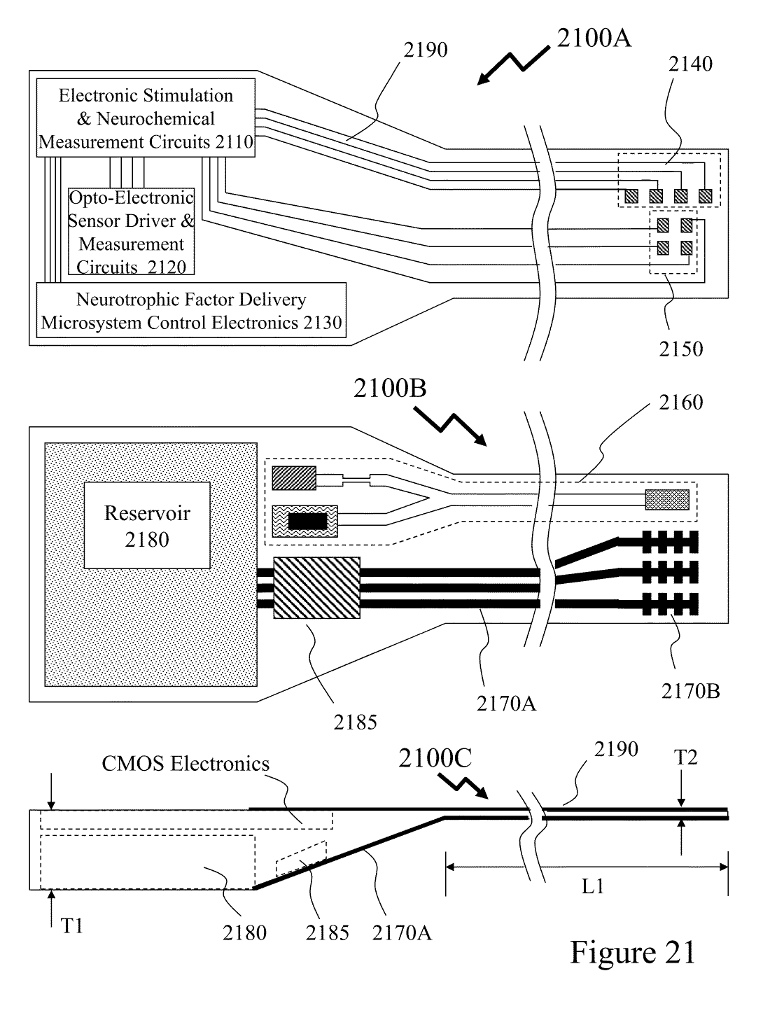

Now referring to FIG. 21 there is depicted an exemplary probe configuration presented as top view 2100A, bottom view 2100B, and side elevation 2100C comprising a neurotrophic factor delivery microsystem according to an embodiment of the invention in conjunction with an optoelectronic sensor and electronic stimulation and neurochemical measurement circuits. Accordingly top view 2100A comprises electrical stimulation site 2140 and neurotransmitter sensor site 2150 which are coupled to Electronic Stimulation & Neurochemical Measurement Circuits 2110 and implemented in 0.18 μm CMOS for example. The Electronic Stimulation & Neurochemical Measurement Circuits 2110 are also coupled to Neurotrophic Factor Delivery Microsystem Control Electronics 2130 and Opto-Electronic Sensor Driver & Measurement Circuits 2120.

The Neurotrophic Factor Delivery Microsystem Control Electronics 2130 are coupled to opto-electronic sensor circuit 2160 whilst Opto-Electronic Sensor Driver & Measurement Circuits 2120 is coupled to micro MEMS pump 2185. Micro MEMS pump 2185 being disposed within fluidic microchannels 2170A that are coupled to the neurotrophic dispensing site 2170B and neurotrophic factor reservoir 2180. The neurotrophic dispensing site 2170B, neurotrophic factor reservoir 2180, micro MEMS pump 2185, and fluidic microchannels 2170A being disposed on the bottom of the probe as shown in bottom view 2100B. Now referring to side elevation 2100C the probe is shown as being of a first thickness, T1, at the end comprising the electronics and reservoir 2180 and of reduced thickness, T2, at the end with the measurement sites, optical sensor, and neurotrophic factor delivery site. Accordingly in this embodiment of the invention the reservoir 2180 is provided within the body of the probe rather than as disposed externally as described supra in respect of FIG. 20. The variable surface geometry of the bottom side of the silicon establishes some additional limitations on the photolithographic and other manufacturing processes employed in manufacturing the microfluidic channels, optical sensor, neurotrophic factor delivery site, and micro MEMS pump. However, in most instances the processes required for these structures due to their geometries are typically provided through manufacturing processes such as 0.35 μm, 0.6 μm, and 1.0 μm which are provided by a CMOS foundry capable of providing mixed circuits comprising analog circuits, digital circuits, and MEMS devices. Alternatively, fabricated CMOS wafers may be transferred to another foundry for the backside processing. According the requirements of the optical sensor it is anticipated that the optical emitter and optical detector would be pick-and-place components provided onto the probe upon completion and verification of the required functionality.

Specific details are given in the above description to provide a thorough understanding of the embodiments. However, it is understood that the embodiments may be practiced without these specific details. For example, circuits may be shown in block diagrams in order not to obscure the embodiments in unnecessary detail. In other instances, well-known circuits, processes, algorithms, structures, and techniques may be shown without unnecessary detail in order to avoid obscuring the embodiments.

Implementation of the techniques, blocks, steps and means described above may be done in various ways. For example, these techniques, blocks, steps and means may be implemented in hardware, software, or a combination thereof. For a hardware implementation, the processing units may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above and/or a combination thereof.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A method comprising:
determining a concentration of a neurotransmitter in-situ using an electrochemical sensor integrated into a probe;
coupling the output of the electrochemical sensor to a CMOS processing circuit integrated with the probe, the CMOS processing circuit providing an output in determination of at least the output of the electrochemical sensor and a reference;
coupling the output of the CMOS processing circuit to a microfluidic delivery system integrated within the probe, the microfluidic delivery system providing localized delivery of a predetermined drug in dependence upon the output of the CMOS processing circuit.

2. A method according to claim 1 wherein;
the electrochemical sensor comprises at least a current conveyor to establish a voltage between at least a pair of sensor electrodes, the voltage generated being dependent upon the concentration of the neurotransmitter.

3. The method according to claim 1 wherein;
the CMOS processing circuit comprises at least a comparator and a latch; and
the reference is a reference voltage determined in dependence upon the minimum acceptable level of the neurotransmitter.

4. The method according to claim 1 wherein;
the CMOS processing circuit comprises an N-bit Delta-Sigma analog-to-digital converter, wherein N is an integer, N>1, and
the reference is a reference voltage determined in dependence upon the minimum acceptable level of the neurotransmitter.

5. The method according to claim 1 wherein;
the electrochemical sensor, CMOS processing circuit, and microfluidic delivery system are all formed upon the same silicon substrate.

6. A method comprising;
maintaining a neurotransmitter above a predetermined concentration with a predetermined region of a brain using a closed-loop neurotrophic factor delivery and control system integrated upon a probe formed from a single silicon substrate.

7. The method according to claim 6 wherein the closed-loop neurotrophic factor delivery and control system comprises at least:
an electrochemical sensor integrated into the probe for determining a concentration of a neurotransmitter in-situ;
a CMOS processing circuit integrated into the probe providing an output in determination of at least an output of the electrochemical sensor and a reference; and
a microfluidic delivery system integrated within the probe, the microfluidic delivery system providing localized delivery of a predetermined neurothropic factor in dependence upon the output of the CMOS processing circuit.

8. A method according to claim 6 wherein;
the closed-loop neurotrophic factor delivery and control system comprises at least an electrochemical sensor comprising at least a current conveyor to establish a voltage between at least a pair of sensor electrodes, the voltage generated being dependent upon the concentration of the neurotransmitter.

9. The method according to claim 6 wherein;
the closed-loop neurotrophic factor delivery and control system comprises at least a CMOS processing circuit comprising at least a comparator and a latch integrated into the probe providing an output in determination of at least an output of the electrochemical sensor and a reference voltage determined in dependence upon the minimum acceptable level of the neurotransmitter.

10. The method according to claim 6 wherein;
the closed-loop neurotrophic factor delivery and control system comprises at least a CMOS processing circuit comprising at least an N-bit Delta-Sigma analog-to-digital converter, wherein N is an integer, N>1; and
a reference voltage employed within the N-bit Delta-Sigma analog-to-digital converter is determined in dependence upon the minimum acceptable level of the neurotransmitter.

11. A probe comprising:
an electrochemical sensor for determining a concentration of a neurotransmitter;
a CMOS processing circuit electrically coupled to the electrochemical sensor providing an output in determination of at least the output of the electrochemical sensor;
a microfluidic delivery system coupled to the CMOS processing circuit for providing localized delivery of a predetermined drug in dependence upon the output of the CMOS processing circuit.

12. The probe according to claim 11 wherein;
the electrochemical sensor comprises at least a current conveyor to establish a voltage between at least a pair of sensor electrodes, the voltage generated being dependent upon the concentration of the neurotransmitter.

13. The probe according to claim 11 wherein;
the CMOS processing circuit comprises at least a comparator and a latch; and
the reference is a reference voltage determined in dependence upon the minimum acceptable level of the neurotransmitter.

14. The probe according to claim 11 wherein;
the CMOS processing circuit comprises an N-bit Delta-Sigma analog-to-digital converter, wherein N is an integer, N>1, and
the reference is a reference voltage determined in dependence upon the minimum acceptable level of the neurotransmitter.

15. The probe according to claim 11 wherein;
the electrochemical sensor, CMOS processing circuit, and microfluidic delivery system are all formed upon the same silicon substrate.
